(19) United States
(12) Patent Application Publication
(10) Pub. No.: US 2013/0253284 A1
(43) Pub. Date: Sep. 26, 2013

(54) Field Medical Reporting System

(71) Applicants: Israel Fraier, Lehavim (IL); Alexander Schwartz, Haifa (IL)

(72) Inventors: Israel Fraier, Lehavim (IL); Alexander Schwartz, Haifa (IL)

(73) Assignee: OMIND LTD., Lehavim (IL)

(21) Appl. No.: 13/819,757

(22) PCT Filed: Sep. 1, 2010
(86) PCT No.: PCT/IB2010/002161

§ 371 Date: Feb. 28, 2013

(51) Int. Cl. A61B 5/00

(52) U.S. Cl. CPC A61B 5/7465
USPC 600/300

(57) ABSTRACT

Device to be attached to a wounded person for recording and reporting medical and logistic information relating to the wounded person in mass casualty event is disclosed, the device is battery free and comprises (i) a solid state memory; (ii) a plurality of actuators in a predetermined correlation with a graphical presentation presented on external surface of the device such that a status of each actuator represents a medical or logistic information relating to the wounded person; (iii) a conversion adapter configured to translate a status of the actuators into respective digital codes or to transfer the status of the actuators to external real time translator for returning the status in the form of respective digital codes wherein said codes represent medical or logistic information relating to the wounded person and are respectively loaded into the solid state memory by the conversion adapter or by the external translator; and (iv) an interface configured to communicate with a mobile electrically powered communication apparatus for extracting electrical energy required for tracing the status of the actuators, for translating it the into the digital codes and for loading them into the memory; said codes are thereby readable by the mobile communication apparatus directly from the memory for being communicated to a remote server, wherein the status of the actuators respective of the codes and in correlation with the graphical presentation presented on external surface of the device allows for immediate interpretation of the status by a human reading of the device irrespective whether the device is energized.

### FIELD OF THE INVENTION

The present invention is in the field of recording and reporting data relating to the medical status of a wounded person.

### BACKGROUND OF THE INVENTION

The first days and especially the first hours/of a disaster event (hereinafter Mass Casualties Event (named also Mass Casualties Incident) and in short MCE (or MCI) are characterized by lack of information from the arena of disaster on wounded and on casualties. A simple tool capable of providing rescue managers of an MCE with the exact location of wounded persons and casualties, and which can enable to build a GIS picture of the medical and related logistic needs in a disaster area, can be of great value.

Emergency medical personnel including Field Medics as Emergency Medical Teams (EMTs), paramedics, and trauma physicians, acting in field scenarios are unfortunately not equipped with a simple to use tool for reporting a wounded person's condition, location, medical interventions performed on him and their approximate time. It is appreciated that this results from the absence of a reporting device which may satisfactorily be configured to carry said reporting tasks and to become standard equipment accompanying emergency medical personnel as a norm.

Medical reports accompanying a wounded person on his way to field emergency centers and to the hospital should be easy to read by other medical staff and should be easily updatable with data relating to additional interventions and locations relating to the wounded person.

Emergency medical personnel acting in field conditions in military battle conditions and/or in mass casualty disasters, accidents, terror attacks and other unfortunate incidents would consider allocating minimum of time, labor and attention for the reporting task. This makes “simplicity of use” a critical requirement to be complied with by an intended reporting tool.

Apart from being simple to use, an intended emergency medicine reporting-tool must work reliably in harsh field conditions and should withstand prolonged storage durations in emergency depots.

Information of the accurate location of the wounded and casualties is important for planning and sending medical transportation means and logistics for the wounded, and identification of casualties. Accurate location that can be provided even in built urban areas e.g. using an EGNOS or WAAS enabled (both are types of augmented Global Navigation Satellite System) GNSS receiver, will enable estimating the disaster dimensions, planning evacuation needs, and optimizing wounded transportation, and medical and logistic support to the area.

US US2007194099 refers to a tag to be attached to casualties, for recording and reporting their personal and medical status. Data to be manually recorded on the tag is visual to the medical personnel and comprise bar code labels which facilitate loading information from the tag to a computerized system. The data appears on both sides of the tag and therefore the tag should be turned over in order to allow for reading information from both sides by a bar code reader. Another drawback of the tag is that different details to be recorded on the tag require different barcode labels to be attached on the surfaces of the tag. It is accordingly required to prepare in advance machine readable data which should then be attached to or removed from different locations on the tag, as a means of recording a detail. In turn, when scanning the information, the entire surfaces of the tag must be scanned by a bar code reader in order to scan, translate and interpret the separate meaning of each barcode label attached to or absent from the tag. Another drawback is that said tag taken stand alone (without being read after each medical check or intervention—a common most probable situation in battle or disaster conditions) doesn't provide the capability to differentiate between the wounded status and interventions in different successive medical check sessions, an important piece of information regarding the medical status outcome direction and type of intervention needed.

It is accordingly an aim of the present invention to provide for a tag for recording and carrying medical information and sequence thereof relating to casualties in mass casualties events, which will facilitate the process of loading information from the tag to a computerized system yet without complicating the process of inputting data to the tag in field conditions and without reducing the readiness of the tag for immediate use in emergency, after years of shelf life.

### SUMMARY OF THE INVENTION

The invention relates to device to be attached to a wounded person for recording and reporting medical and logistic information relating to the wounded person in mass casualty event, the device (hereinafter Medical Status Device or MSD in abbreviated form) is battery free and comprises (i) a solid state memory; (ii) a plurality of actuators in a predetermined correlation with a graphical presentation presented on external surface of the device such that a status of each actuator represents a medical or logistic information relating to the wounded person; (iii) a conversion adapter configured to translate a status of the actuators into respective digital codes or to transfer the status of the actuators to external real time translator for returning the status in the form of respective digital codes wherein said codes represent medical or logistic information relating to the wounded person and are respectively loaded into the solid state memory by the conversion adapter or by the external translator; and (iv) an interface configured to communicate with a mobile electrically powered communication apparatus for extracting electrical energy required for tracing the status of the actuators, for translating it the into the digital codes and for loading them into the memory; said codes are thereby readable by the mobile communication apparatus directly from the memory for being communicated to a remote server, wherein the status of the actuators respective of the codes and in correlation with the graphical presentation presented on external surface of the device allows for immediate interpretation of the status by a human reading of the device irrespective whether the device is energized.

The invention further relates to a mobile electrically powered communication apparatus (hereinafter DRI) configured to communicate with the MSD for temporarily energizing it and for exchanging data with the memory of the MSD.

A medical recording displaying and reporting system for displaying and electronically storing a status of a wounded person in a mass casualty event, is also concerned, the system comprises a remote server capable of receiving data from the memory of each of a plurality of MSDs through at least one mobile communication apparatus.

As will be further explained in detail, the actuators of the invented MSD respond to manual actuation by electrically measurable change in resistance, conductivity, inductance or capacity or a combination thereof of respective sensors embedded in a body of the MSD.

According to various embodiments of the invention the actuator comprises chemical reactants configured for changing the resistance of a respective sensor simultaneously with making a change in color visible in a predetermined location within the graphical presentation presented on external surface of the device, said change in color is indicative of a change in the status of the actuator.

The actuators according to some preferred embodiments of the invention may comprise metal foil attached on an external surface of the MSD, each having a predetermined shape configured to be peeled off the surface for making electrically measurable change associated with medical or logistic information relating to the wounded person and for simultaneously making on a surface of the device a visible or tactile human interpretable change indicative of a change in the status of the actuator.

According to some embodiments of the invention the actuators comprise scratch off coating in predetermined shapes and locations within the graphical presentation presented on external surface of the MSD, the scratch off coating has predetermined conductivity or comprises reactants in microcapsules adapted for causing electrically measurable change in resistance, conductivity, inductance or capacity or a combination of thereof of respective sensors embedded in a body of the device, once scratched off the surface of the MSD, and simultaneously making on the surface a visible or tactile human interpretable change indicative of a change in the status of the actuator.

According to yet further embodiments of the invention the actuators comprise a conducting rubber plug to be pulled out of the device.

In various preferred embodiments of the invention the actuators comprise a plug to be pulled out of the device, a foil to be peeled off a surface of the device or a scratch off coating to be scratched, said actuators are opaque to ambient light, wherein a photoelectric element (e.g. photoresistor, photodiode, phototransistor or any other device which changes its electric properties when exposed to light) is located under each of at least part of a plurality of the actuators, and a substantial change in its conductivity is detectable once the respective actuator is removed from above.

According to a preferred embodiment of the invention the actuators are disposed on the graphical presentation in repeating order, each order is associated to a same predetermined list of typical medical interventions which may be taken during a treatment of a wounded person, thereby allowing the evolving status of a wounded person over successive intervention to be visible and appreciable directly from the MSD without requiring a reading apparatus.

It is appreciated that in case the transmitting and receiving range of the MSD is increased, the communication apparatus (DRI) will be able to communicate with and retrive information from the memories of a plurality of MSDs attached to different wounded persons, e.g. for reporting their medical status to a remote server, without the need to come near each MSD separately. Accordingly, in some embodiments of the invention the MSD may further configured for mutual plugging with a with a powered module (e.g. Active RFID) comprising wireless transceiver (e.g. IEEE 802.15.X, UHF RFID, Sub-Ghz ISM—Industrial, Scientific and Medical or other) and a power source (e.g alkaline button cell battery) and associated communication control circuitry (e.g. Integrated communication controller, integrated CPU or a like), said module (referred to also in this invention “communication booster module”) is thereby may optionally be connected to the MSD's CPU serial communication interface (e.g. SPI—Serial Peripheral Interface, I2C—Inter Integrated Circuit, 1-Wire Bus, Microwire, UNI/O or a like) in case an increasing communication range with the DRI is required on site, in case an increasing communication range with the DRI is required.

The invention relates also to a method for complying with medical triage, logistics and casualties transportation decisions demands associated with the managing of medical support of wounded persons in mass casualty event, the method comprises equipping medical staff with (i) a plurality of medical status devices (MSDs) configured to allow for manual recordation of respectively correlated human readable and electronically traceable changes relating to medical interventions performed on or to medical measurements taken from the body of a wounded person, for accomplishing the recording of the casualties' medical status; and with (ii) at least one reading and writing device DRI configured to read information recorded on the medical status devices and to return information to enrich the information recorded on the MSD, wherein said medical status devices are attached each to a wounded person in the MCE, thereby allowing a medic treating the wounded person to record interventions and medical measurements taken to browse through the information recorded on the MSD either electronically by the DRI or visually directly from the physical status of actuation sites located on surface of the MSD. Preferably the DRI further comprises communication means or is capable of exchanging information with a mobile communicator in communication with remote server, thereby allowing to report the information recorded in MSDs to a remote server, thus improving the ability to comply with logistic demands associated with the managing of medical support and transportation of wounded persons in the mass casualty event.

The invention relates to a battery free Medical Status Device (MSD) and to a concept and method of use of a Medical Field Recording displaying and Reporting System (MFRS) directed to enable a field medic/paramedic/EMT (Emergency Medical Teams) to use the said MSD as a user interface for manual hardware recorded inputting, and displaying and for electronically storing a status of a wounded person. The status may include e.g. wound symbolic description, physiological parameters levels, and medical interventions/procedures applied to the wounded person. After the status is manually recorded on the MSD, it may later be automatically converted and stored in said MSD on an electronically readable memory, once the MSD is temporarily energized by external source of energy. Information from the memory may be read and further transmitted by a DRI unit (Data Reader, Input and communication relay) which may be utilized also as said external temporary source of energy. The stored status may be enriched upon reading with data relating to the last known location, time of reading and ID associated with said DRI, as well with the ID of the personnel in charge or any other information or sensor data collected or inputed into said DRI. According to one embodiment it is assumed that the field medic will report the status of a treated wounded person by the DRI unit immediately after placing an MSD on the wounded person. Accordingly, said ‘time of reading’ may later on serve for automatically calculating the lapse of time since said medical intervention, the TFI (Time From Intervention).

The MFRS of the present invention will be used by field medics for the purpose of reporting and informing the medical staff of next levels of medical care (e.g. field trauma centers, field hospital, regular hospital, etc.) and disaster and Mass Casualties Event (MCE) managers on a wounded person's status, TFI and location, wherein advantageously the reporting is made both visually (or otherwise tactile) and electronically, directly from the field, by the MSD accompanying the arriving wounded person, thereby enabling medical resource allocation and transportation to the most suitable hospital facility.

The MFRS of the present invention comprises (i) a fettered or otherwise attachable battery free electronically readable MSD to be secured to a member e.g. limb or neck of a wounded person; (ii) a powered handheld DRI for reading said MSD, and for loading additional data such as time, location and medical status data into the memory of said MSD via wired or RF (radio frequency), Near Field Communication (NFC). According to various preferred embodiments of the invention the DRI is a commercial third party mobile communicating device, e.g. a USB Host/OTG enabled cellular/WiMax/WiFi/phone or the like, connectable to the MSD by USB or NFC (if said phone is provided with NFC means), and alternatively is an equivalent custom built communicator e.g. a custom built or custom adapted military communication terminal or the like.

More particularly the MSD according to the present invention is a battery-free device, and is configured for intuitively inputting and visually and/or by tactile means displaying the status of a wounded person. Said intuitive inputting and displaying are achieved by providing a series of actuation sites embedded in the device or on its surface, wherein said sites are configured such that a predetermined physical manipulation performed on an actuation site of the series will result in modification/s in the device which is/are both immediately interpretable by a human and automatically convertible into electronic data to be stored in a memory comprised in the MSD once the MSD is temporarily energized be external power source. In various embodiments of the invention the physical manipulation may be provision of one of (i) mechanical pressure; (ii) rupture; (iii) peeling; (iv) puling out a plug; (v) light illumination (either by exposure to ambient light or to active illumination); (vi) electrical contact; thereby causing a change in physical or chemical property of the site. An immediately human interpretable sign may be achieved by using a physical manipulation such as uncovering color spots, modifying texture, releasing chemicals capable of providing a change of color, or the like, while simultaneously the manipulation results in modification in the recorded automatically convertible content of the MSD to be stored electronically in its memory once powered, which is thereby becomes readable by said DRI.

Simple and fast method for reporting wounded person's medical status and field interventions, approximate lapse of time from each of said medical interventions, location of the wounded person, to the next level of medical care and MCE managers is thus provided.

Taking in account the advantageous display and input capabilities of the MSD of the present invention and the automation of time and location data inputting provided by its interaction with a DRI, it can be stated with great confidence that once NFC standardization of cellular phones will be completed as expected, the MSD of the present invention in cooperation with said standard NFC means, will enable any such NFC cellular (or WiFi, and the like.) phone provided with a suitable software application to serve as a DRI, thereby saving organizations dealing with MCE (Mass Casualties Event) the expenses of purchasing dedicated DRI units.

The MSD according to the present invention is a battery-less (i.e. free of electrical battery) unit which does not require electrical power source for recording electrically traceable indications relating to the medical status of the wounded person. The MSD comprises a solid state memory and an integrated circuit configured to convert the electrically traceable indications into digital information and to store it in the solid state memory. Once a DRI is coupled to and temporarily energizing the MSD, the integrated circuit and the solid state memory are powered and the traceable indications may be traced converted and stored. The DRI can then read the medical status of the wounded person directly from the solid state memory of the MSD despite of the lack of energy source at the time the traceable indications have been inputted. Simultaneously, supplemental data may be uploaded to the solid state memory of the MSD from the DRI and its terminals, for the benefit of medical teams which will take care of the wounded person at later stages. The MSD comprises (i) an intuitive user interface having fast and easily hand operable series of actuator means located at predetermined sites in the device the physical manipulation of which resulting in human observable modifications of the MSD device. An immediately human observable and interpretable sign may be achieved by making use of a physical manipulation such as uncovering color spots, modifying texture, releasing chemicals capable of providing a change of color, or the like, while said manual manipulation is simultaneously modifying the automatically convertible content of the MSD. The physical manipulation to be used may be based on provision of means which respond to e.g. mechanical pressure, rupture, peeling, pulling out a plug, light illumination (either by exposure to ambient light or to active illumination), changing an electrically measurable properties such as resistance, conductance, inductance, capacitance, or the like, by simultaneous changes in physical properties at least one of which is immediately interpretable by a human and at least one of which is electrically traceable and convertible.

Said physical intuitive user interface is configured to provide a twofold function in response to a physical manipulation performed by the user, the twofold function comprises providing a predetermined human interpretable display associated with a respective physical manipulation performed and simultaneously providing a predetermined automatically convertible recordation in association with the respective human interpretable display. The human interpretable display may be a user observable change e.g. in the color, exposing fluorescent background, or pattern, or structure of said marked areas for fast and easy recognition of the change and its interpretation as a visual information report of the medical status and the interventions applied to a wounded person carrying the MSD. Changing the automatically convertible content of the MSD by the physical manipulation is advantageous in that it requires no electrical energy thereby allowing the MSD to be stored unlimited periods until an emergency event occurs, while allowing a recorded information to be read electronically once a DRI (either dedicated device or an NFC or otherwise connectable e.g. USB phone) is available and connected to said MSD device, thereby enabling later electronically memorization of the report of the medical status and the interventions applied which in turn will allow for transmitting these details along with all previous reports stored in said MSD memory, via communication means to the next level of medical care, and MCE managers. The MSD further comprise energy consuming components to be temporarily energized by a DRI interacting with the MSD, such components include (i) a memory e.g. solid state memory for recording the medical status report on the wounded person, said memory is preferably being protected (e.g. by software locking which demands a code to be written to the memory prior to changing any contents or by hardware discrete pin logic level) from erase (e.g. unintentionally) by an MFRS user, (ii) a CPU (Central Processing Unit) and respective analog frontend electronics for measuring the automatically convertible variations generated by the reporting user, and for controlling the communication between said memory and the DRI for data exchange purposes; (iii) an electronic displaying means e.g. LEDs to indicate that the device is in data exchange with the DRI; (iv) an input port for connecting said DRI (including its power and data transfer means); (v) a wired or RF NFC contact-less power and data transfer means for receiving power and exchanging data from a DRI, wired or optionally provided with NFC contact-less power and data transfer means (or from an NFC provided cellular phone in its role as DRI); (vi) a fetter or other attachment means configured for securing the MSD to a member e.g. limb of the wounded person or for otherwise attaching said device to the wounded person.

Automatically convertible change resulting from physical manipulation of said actuator can be achieved for example by configuring the actuator for interrupting a circuit path or for varying the resistance, conductivity, inductance, or capacitance, of a sensor site, in response to the manipulation. The change is then detected, measured and converted to digital binary code by the CPU of the MSD as a change in voltage, current or other electronically measurable value.

In various embodiments of the MSD according to the invention, a capacitive/inductive sensor is provided as a means for translating the physical manipulation into automatically convertible recordation. No electrical power is required for changing a capacity or inductivity to be sensed later once a DRI is in cooperation with the MSD making electrical power available. In preferred embodiments the capacitive or inductive sensor is etched on a PCB (hard or foil). The actuator-like effect resulting from a physical manipulation in these embodiments may be obtained by a change in the distance or in the presence of certain material (e.g. an aluminum foil in case of an inductive etched sensor, or dielectric material in case of a capacitive etched sensor) in the proximity of said sensor.

The change in the capacity or inductivity resulting from the physical manipulation is measurable by a power consuming circuit consisting of an AC voltage generator connected (through a set of electronic switches) to the sensor. Once electrical power is available (i.e. upon wired or RF powering and communication with a DRI), the AC voltage may be connected to the sensor by CPU instructions, a change in the inductivity/capacity of the sensor will thus be measured as a voltage change, which in turn will be translated by the CPU (or by external real time translator receiving data from the CPU) into a binary code value which is loaded into the memory of the MSD. In a similar manner, a resistance/interruption based sensor may be implemented in yet further embodiments of the invention, in which by e.g. pulling out a conducting rubber plug a circuit path in a printed board is either interrupted or is changing its resistance. Alternatively said pulling out of a plug or peeling of a cover pellicle or scratching it out will expose to ambient light a photoelectric element (e.g. photoresistor, photodiode, phototransistor or any other device which changes its electric properties when exposed to light) preferably sensitive to NIR (near infrared light) to be usable also with night ambient light, NIR, generating a substantial change in its conductivity to be detected once the respective actuator is removed from above e.g. by measuring the change of resistance using conventional Wheatstone bridge circuit. A change in the sensor will then be detected (i.e. once electrical energy is available upon interaction with a DRI) by any conventional IO controller and in turn will be translated by the CPU into a binary code. Advantageously, all values read from an MSD together with the MSD ID number and optionally together with time and location data from the DRI and its user ID identification will be packaged by the DRI software application into a time identified status report, to be loaded into the MSD memory and optionally transmitted via communication means to the next level of medical care, and MCE managers.

The DRI according to the present invention is a battery powered unit comprising (i) an RTC (Real Time Clock) date and time measuring circuit (herein after “AT”) for automatically reporting the date and time of reading of said medical status report, from and into the MSD; (ii) control means as part of the DRI's CPU and its working memory, for controlling certain DRI units and for calculating the TFI (time from intervention) by calculating the time difference between the AT present time measured by the DRI and the AT of the medical reports stored in the MSD memory; (iii) (optionally) a display element for displaying the medical status report number and its TFI time, for informing the medical staff of next levels of medical care on the time that has passed from a particular medical intervention e.g. infusion, applying a tourniquet etc.; (iv) a read/record button for reading the said MSD medical status data and serial ID number (which may serve as a temporary identification number of the wounded person to be associated with his official ID once available), and recording it into said MSD memory together with the AT and GPS data of this medical intervention session; (iv) computational means (as a part of the DRI's CPU) and control electronics with respective activation buttons for browsing the medical status reports recorded in the MSD according to their TFI, said control electronics and buttons may be used for e.g. choosing to play back changes in a particular report with respect of a previous report (e.g. interventions added after the previous report), said control buttons will preferably provide back and forward control capabilities and will preferably include a dedicated button for fast back-to-first record (highest TFI); (v) (optionally) a GPS unit for reading the DRI geographic coordinates and GPS AT and recording these into the MSD memory, optionally said unit may be programmed to initiate a read of its GPS data at turning on the DRI unit and thereafter at preprogrammed time intervals, or upon connecting the DRI to the MSD. The advantage of a built in GPS unit, preferably a high precision EGNOS/WAAS (augmented global navigation satellite system) enabled unit, being twofold: (a) providing exact location data of a wounded person at the time of a particular medical status report; (b) providing absolute time, from the GPS data, usable for updating and calibrating the built in RTC unit and saving field medics the responsibility and need to update the DRI with accurate date and time; (vi) an optional RF NFC (Near Field Communication) contact-less power and data transfer means by which the DRI (I) transfers power to the MSD using a tuned antenna coil and (II) records and extracts data to and from the memory of the MSD, preferably using an NFC protocol. Each of said MSD and DRI may be provided with an optional wired connection (stretchable cord) for connecting the DRI to the MSD, wherein preferably the length of the cord in its fully stretched form is about 2 meters, in order to comply with difficulties which may rise from the position of a wounded person position, or which otherwise may occur in MCE; (vii) an input port for connecting an external wireless e.g. cellular, military radio, WiFi, Bluetooth, etc., transmission means (herein after “TM”) for connecting reading and sending the medical status records including their AT, calculated TFI, and GPS location stored in the MSD memory to a remote server, or a built in TM mean e.g. cellular, military radio, WiFi, Bluetooth, etc., for sending to a remote server, the medical records, AT and GPS data once said MSD records have been read from its memory by said DRI. A cellular modem enabled DRI unit can be easily built by using for example a combined GPS-GPRS module e.g. GM862-GPS modem made by Telit, or other commercial GPS-cellular modem modules. The DRI to server transmission can be automatically activated by the DRI to MSD read/record button or alternatively by a dedicated send button disabling unintentional transmission of reports.

In yet further preferred embodiment the DRI comprises memory of a size sufficient for capturing a large number of status reports (for example several hundred reports) from a plurality of MSDs, including their serial ID's and their AT and GPS data. The DRI may thus record in its memory a backup of all reports recorded in all MSDs placed on a number of wounded persons by said field medic in a time period.

Such backup may be useful e.g. for dispensing the need to repeat reading MSD reports in case of communication problems.

In an optional alternative embodiment the DRI comprises at least one user input means e.g. a typing keyboard input means, or a microphone voice input means, an imaging camera, or the like, to be used by said field medic for reporting and recording additional relevant data, e.g. his own ID number, exact physiological values of the wounded person, etc., into said DRI for transferring it into the MSD's memory via wired or RF e.g. NFC communication means, as well as to a remote server of the MCE disaster centre. In such an alternative embodiment the DRI may optionally comprise a speaker/headphone e.g. for receiving voice instruction about the location and route leading to a wounded person.

In embodiments using an NFC provided phone as a DRI, all said input, display and playback means may be the built in phone's means operated by a MFRS dedicated software application running in the NFC phone.

The MFRS and it's method of operation as conceived in the present invention present a simple and reliable way to report the wounded person's medical status and field interventions and the approximate time that has passed from each of said medical interventions, to the next level of medical care. The information is provided both with the arriving wounded person as a human readable data, and electronically as transmitted directly from the field for enabling medical resource allocation and transportation to the most suitable hospital facility. An example of using the MFRS of the present invention can be envisioned as follows:
1. The field medic is provided in his first-aid kit bag with a multitude of MSD units and one or two DRI units.
2. Before entering the emergency or battle arena, the field medic will start the DRI and check its battery status by pressing the DRI “on” button, which optionally will also automatically activate the GPS unit and record a location fix.
3. The field medic will optionally type-in his personal ID to be transmitted and recorded into said MSD, using a numerical keyboard of the DRI.
4. On reaching the emergency arena or battle field, the field medic will short press the read/record button for updating the location data in case the GPS is not taking measurements automatically.
5. On reaching a wounded person and after performing first aid emergency procedures, the field medic will take an MSD device from his first aid bag, manipulate the necessary actuator-like sites according to the wounded person status and the interventions taken, e.g. by peeling a cover foil, tearing, pressing, pulling out a plug, puncturing the site, etc., attach/fetter the MSD to a member or neck of the wounded person, and connect it for a moment to the DRI using the NFC proximity contact or coiled connection cord. The DRI will automatically read the MSD and update its memory and optionally transmit the data to the medical and emergency system via its communication means.
6. If the said Field Medic or one of the next level of medical care, will need to report an additional procedure taken, he will physically update the MSD's actuator-like sites and repeat the previous procedures. The DRI (or the NFC phone) software application will extract the differences between the current MSD sensor's status and the previous report and generate a new time marked report updated with the last medical interventions done to the wounded person.
7. For later transmission of the said wounded person status/interventions to a remote server/medical or MCE authority, the field medic will press the send button if said transmission means are built-in into the DRI, or connect the DRI to external transmission means.

Apart from accurate and timely reports of the wounded person status and approximate time from the reported medical intervention, other important benefits of the MFRS disclosed in the present invention are the automatic identification via the DRI ID or Login ID, of the field medic or medical staff that has treated a particular wounded person, the MSD's ID interim identification of the wounded person or casualty, and the automatic identification of the location the wounded or casualty was evacuated from. Medical emergency services will be able to use the said AT and GPS data of the wounded persons or casualty to create a real time GIS map of the evacuation and casualty recognition needs and allocate resources accordingly.

The most important benefits of the present invention consist in the dual report capabilities of the MFRS system: on the one hand the system enables the transmission of immediate reports from the field on the wounded status, enabling planning of transportation to the nearest suitable hospital and providing in advance information to the hospital on the status of the arriving wounded patient. On the other hand, upon receiving the wounded person, the hospital physician or related staff member will immediately be able to take a look at the MSD device and instantly learn the wounded initial status and the type and number of performed intervention (e.g. number of infusions, number of atropine injections, air way opening, tourniquet application, etc.). In addition, the hospital physician or related staff member will be able to receive a computerized picture of all previous reports relating to the said wounded, add new performed interventions, medical measurements data, etc. all in a computerized data environment for optimal follow up of treatment.

The MSD of the present invention is an ideal practical tool that facilitates seaming between the instantaneous report and read capabilities required by the field medic acting in harsh conditions and between the computerizing requirements relating to medical status data of a wounded patient required by medical staff and hospital physicians in a trauma department, for better treatment of the wounded person.

### BRIEF DESCRIPTION OF THE FIGURES

FIG. 1A illustrates Schematics of the DRI configuration

FIG. 1B illustrates a diagram of MSD according to a first exemplifying embodiment of the invention.

FIG. 1C—illustrates PCB capacitance and inductance sensors according to the invention.

FIG. 1D illustrates PCB resistive sensors according to the invention.

FIG. 1E illustrates a diagram of the MSD according to a second exemplifying embodiment of the invention.

FIG. 2 illustrates electronic block diagram of an MSD according to the present invention.

FIG. 3 illustrates Electronic Block diagram of a DRI according to the present invention.

FIG. 4 illustrates electronic scheme of a circuit for tracing change in capacitance based actuators according to the invention.

FIG. 5 illustrates electronic scheme of another embodiment of a circuit for tracing change in capacitance for capacitance change actuators according to the invention.

FIG. 6 illustrates electronic scheme of a circuit for tracing change in inductance for inductance based actuators according to the invention.

FIG. 7 illustrates electronic scheme of a circuit for tracing change in resistance, useful for resistance and photoelectric based actuators according to the invention.

### DETAILED DESCRIPTION OF THE FIGURES

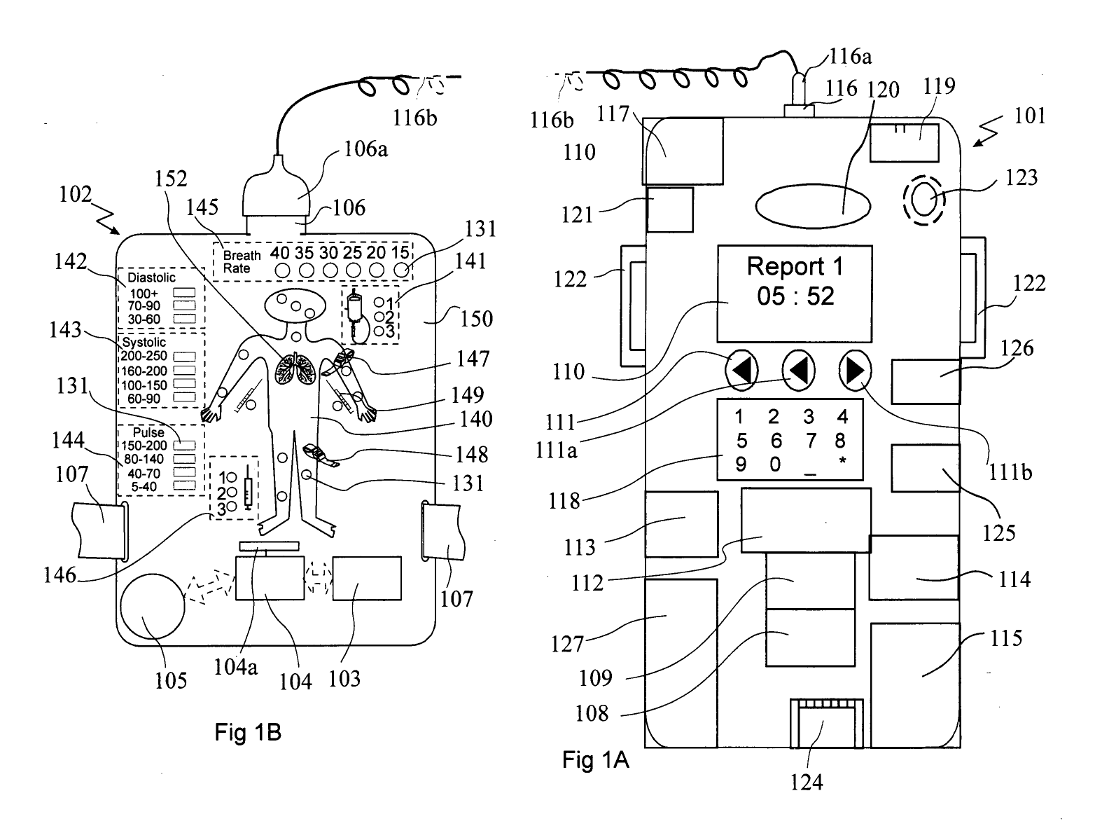

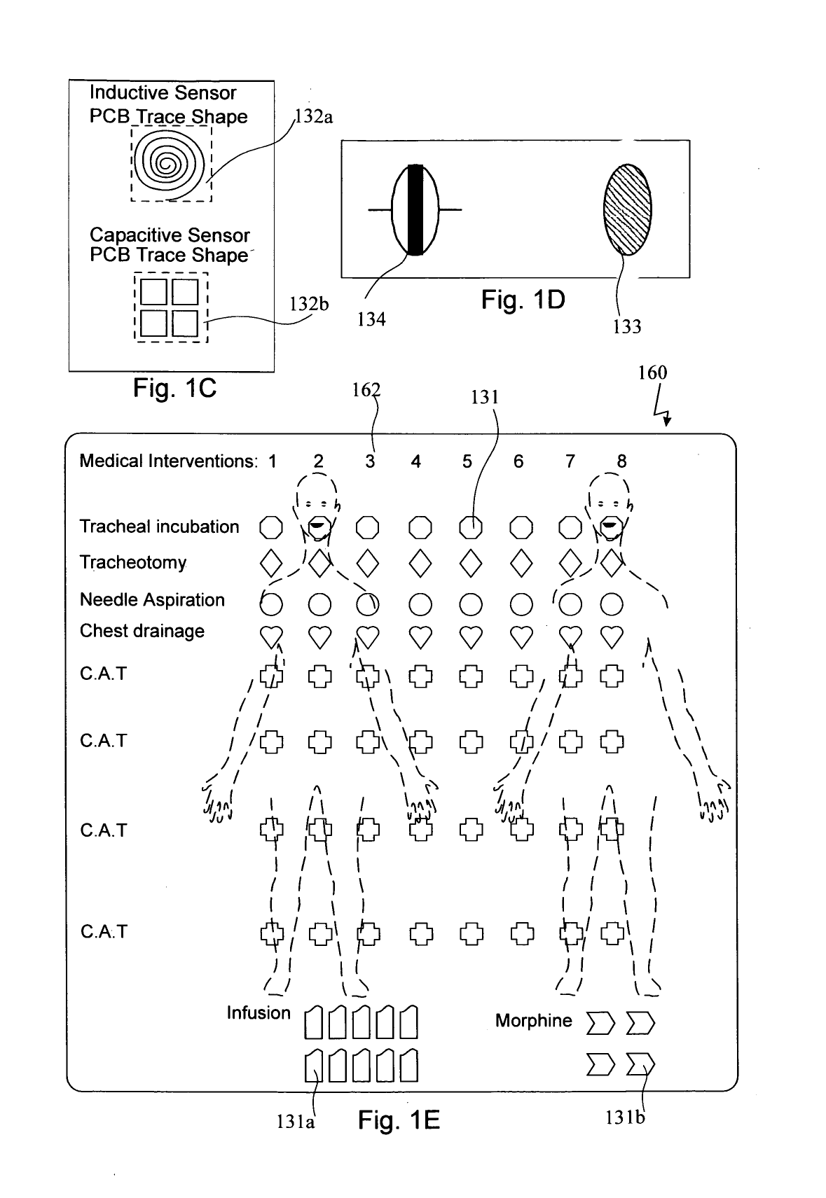

The invention is exemplified with reference to the schematic drawings in FIGS. 1-3, which are not according to scale. The invention having been disclosed, variations will now be apparent to persons skilled in the art, the MFRS system and its units being described as an example only, not to be construed in a limiting way.

In particular once the concept of a battery free device that serves as a physical intuitive user interface for inputting and displaying (visual, tactile, etc.) multi-parameter status information by physical manipulation of a series of device's active or passive actuator-like sites in the device or on its surface, for attaining a double goal of serving as a visual multi-parameter indicator of the said status information on the one hand, and for acting as an electronically readable information source of said multi-parameter status information on the other hand, of this invention having been disclosed, multiple engineering variations battery-less or battery powered, one time switches or switches for multiple use, use of various sensor types, surface or volume sensors or actuators, different shapes of the MSD, different schematic illustrations on the MSD, etc., will now be apparent to persons skilled in the art, all such engineering solutions being also in the scope of the present invention.

FIGS. 1A and 1B illustrate the field devices of MFRS system according to the present invention. The MFRS system comprises a remote server (not illustrated), a plurality of battery free MSD (102) to be attached to wounded persons in a MCE, at least one battery operated DRI handheld terminal unit (101) serving as Data Reader Input and communication relay according to the invention. Each MSD in the system preferably has a unique ID. The ID of the MSD, and the medical status records of a wounded person recorded on a memory of the respective MSD (according to various preferred embodiments of the MSD as exemplified by FIG. 1E, each reading of the medical status together with the Reader's ID, GPS and AT data becomes a separate record in the MSD, thereby enabling next reader to learn and analyze the medical history of the wounded person as it develops) are read from the memory by e.g. RF NFC between the DRI and the MSD. AT and GPS data from the DRI and optionally the ID of the field medic operating it, may be loaded by the DRI into the memory of the battery-less MSD unit (102) to enrich the information stored in the memory of the MSD for later use. On the next MSD reading, and assuming an MSD type allowing multiple recording of interventions is utilized (e.g. the type depicted by FIG. 1E which allows for up to eight successive intervention sessions to be recorded) a field medic will be able to use his DRI for browsing through the previous records recorded in the memory of the MSD and to learn how the medical status of the wounded person is evolving. Advantageously, however, no reading apparatus is required since the actuators of the MSD using as a user interface for the MFRS electronic data recordation and transmittal system allow not only to read and write the recorded information by electronic apparatuses but also to read it by human directly from the graphic design of the surface of the MSD and according to the physical changes affected by the actuators on specific sites on said surface.

The MSD is intended to be secured to a member of the wounded person or casualty, e.g. by a fetter like strap (107) during the first contact between the field medic and the wounded person. The MSD is battery free, and is thus being powered by power transmitted from the DRI during communication with said DRI (101), while simultaneously exchanging data with said DRI via a power and data cord (116b) or via an NFC contact-less power and data transfer protocol. In a preferred embodiment the MSD (102) according to the present invention comprises a solid state memory (103). Preferably means are provided to protect the memory from being erased by users of the MFRS. The MSD further comprises a CPU and memory control (104) for exchanging data between said memory and the DRI for read and record purposes; optional NFC contact-less power and data transfer means (105) or an input port splash and dust proof connector (106) for connecting it to said DRI (preferably when using a wired connection the power and data transfer coiled cord (116b) will be of a max length of about 2 meters (i.e. in its stretched form), in order to avoid difficulties in reaching the wounded person in case he is located or oriented in an inconvenient position. The MSD comprises a fetter like means (107) for securing it to a member e.g. limb, neck of a wounded person.

In this example embodiment of the present invention, the DRI (101) comprises an RTC date and time measuring circuit (108). The DRI further comprises control means as part of the DRI's CPU (109) and its working memory, for controlling certain DRI units and for calculating the TFI (time from intervention). Display element (110) is provided for displaying a serial number of the medical status report and a TFI time thereof. Read/record button (not illustrated) is provided to allow for reading the said MSD data. Computational means as part of the DRI's CPU and associated control electronics and buttons (111)(111a)(111b) are used to allow browsing the medical status reports according to their TFI and for choosing to display a particular report. A solid state memory unit (112) may be used for automatically recording and storing all medical reports and associated data. Preferably the memory is of a size capable of storing a large number of MSDs' reports thereby may serve as a backup to be used in case of communication problems. The memory is useful also for information backup and post-event analysis of emergency situation's problems and performance. GPS unit (113) is preferably provided for reading and recording location and time data. RF NFC (Near Field Communication) contact-less power and data transfer module (114) and antenna (115), and/or alternatively a cord connector (116), are provided, allowing for power and data transfer between the DRI and the MSD. A built in communication module (117) preferably a combined GPS-GPRS module (e.g. GM862-GPS modem made by Telit™, or other GPS-cellular modem commercial modules), is preferably provided, and alternatively a connection to such communication module is provided in order to allow the DRI to communicate with the remote server of the MFRS. The DRI is further provided with input means, preferably a numerical keyboard (118), and is optionally equipped also with imaging camera (119), speaker, (120)/headphone socket (121). These may be useful e.g. for receiving voice instruction on the location and route to a wounded person. A read/record right and left button/handle (122), a LED (123) for indicating whether the MSD is actually communicating, may also be provided in order to facilitate operation of the DRI. An input port (124) for connecting third party equipment using special cable adaptor as connecting to a PC USB port, WLAN, and external wired wireless TM, e.g. cellular, military radio, WiFi, Bluetooth, or the like, may be provided in order to allow for communication between the DRI and a remote server, said transmission to be optionally initiated by the connection of said TM to the DRI, or by pushing a dedicated DRI “send” button (125) as a means for avoiding transmission of unnecessary reports. The DRI is preferably activated by an “on/off” button (126) and is powered by ordinary replaceable or rechargeable batteries (127). The DRI will preferably be built robust and splash proof for field conditions usage.

In embodiments of the present invention using an NFC provided phone in the role of DRI, all said input, display and playback means will normally be the already built in NFC provided phone's means, and will be operating by a dedicated software MFRS application installed in the said NFC phone.

FIG. 1B illustrates an MSD user interface according to the present invention. In this example embodiment the MSD comprises a tablet carrying a schematic human body illustration (140) outlined adjacent to schematic illustrations of relevant physiological parameter values and medical objects (e.g. blood pressure ranges (142)(143), respiration ranges (145), infusion packs (141), injection doses (146), etc.). The said schematic illustrations have tear-able cover patches (131) made of conductive or dielectric foil, the foil is covering respective sensors ((132a)(132b) of FIG. 1C) e.g. etched inductive ((132a) of FIG. 1D) or capacitance ((132b) of FIG. 1C) elements, in various places on the illustrations' extent, each of the sensors or a combination thereof is for expressing a medical report value. Said tear-able cover patches (131) when peeled off, are exposing each a respective color or a fluorescent dye background, or any alternative observable mark, said marks having a display role serving for fast assessment of the wounded person status and performed medical interventions taken by any medical personnel approaching the wounded person. By tearing (in this particular example embodiment) said cover patches the user affect the inductance or capacitance of said inductive or capacitance respective sensor (132), such change leading to a measurable voltage translated by the MSD's CPU (Central Processing Unit) (104) into a binary code value. In an alternative embodiment of the MSD said sensor is resistive and its activation is achieved by pulling out a conducting rubber plug ((133) of FIG. 1D) for interrupting/changing the resistance of a circuit path ((134) of FIG. 1D) of a printed board crossing the sensor's site, said resistance change is detectable by a conventional JO controller (104a) and translated by the CPU (104) into a binary code value which in turn is loaded and stored in the solid state memory (102).

FIG. 1E illustrates the visible surface (162) of another preferred embodiment (160) of an MSD according to the present invention. This embodiment differs from the one illustrated in FIG. 1B in that it comprises a plurality (eight, in this non limiting illustration) of columns, numbered 1-8, each having a plurality of actuation sites (131) to be scratched or pilled off the surface according to medical interventions performed on the wounded person wearing the MSD. The interventions are specified on the left of the surface, allowing a field medic to record the interventions by actuating the relevant ones from the plurality of actuators (131), thereby causing a visible and tactile change on the surface, indicative of the intervention taken. In a first session of interventions the field medic is instructed to use the actuators on the left most column (numbered 1). In successive sessions the field medic will use the actuators in the remaining columns in a respective successive order. Accordingly, the MSD will provide immediate human interpretable status of the interventions taken and of changes occurred during a treatment of the wounded person. The same information (preferably enriched by logistic information received from DRI communicated with the MSD, e.g. GPS, RTC, medic ID and the like) will be stored also in a memory (not shown) of the MSD (160) once the MSD has been energized and the changes in the status of the actuators has electronically been traced and loaded to the memory.

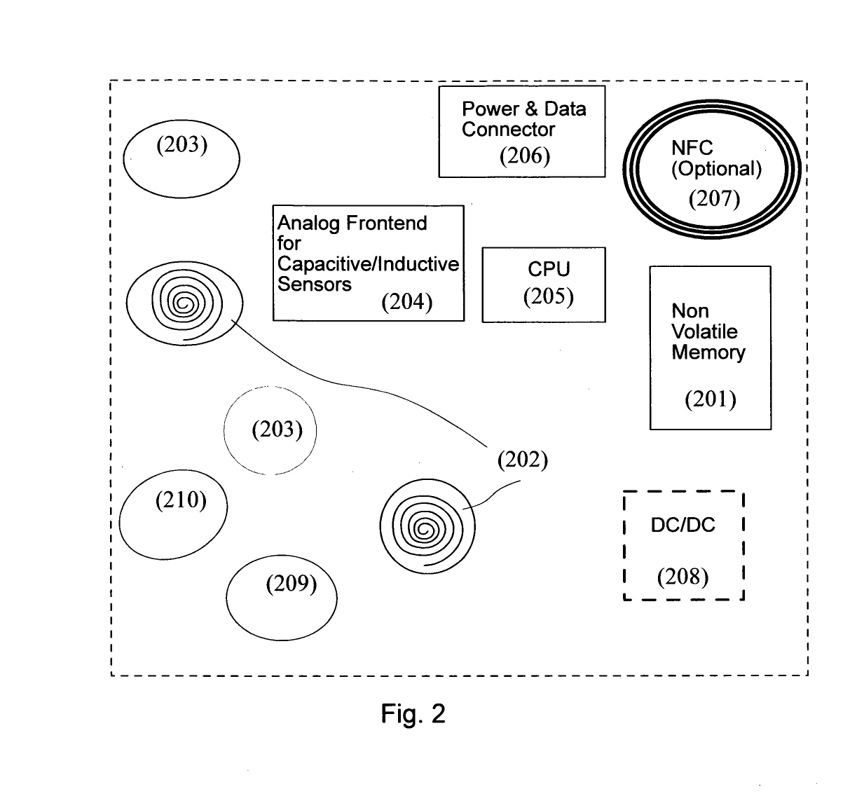

FIG. 2 depicts the Electronic Block diagram of an MSD according to a preferred embodiment of the present invention. All the components presented in the drawing are available from various vendors. The MSD comprises a non volatile memory (201) for storing all the patient status information collected from MSD sensor as well as data transmitted from the DRI such as GPS data, AT time, medic ID etc. Apart from exchanging data with the DRI, this memory (201) will enter “locked” mode against erasure, wherein turning into the locked mode is preferably by hardware, in order to protect it from unauthorized or accidental data loss or corruption.

In this example embodiment the MSD comprises a plurality of etched inductive sensors (202) (only two of which are represented in this Fig.) and a respective plurality of aluminum foil tear-able cover patches (203) (only two of which are represented in this Fig.). The sensor (203) is a part of a printed electronic circuit covering the entire surface of the body of the MSD e.g. a single or double sided printed board tablet carrying said schematic illustrations. The MSD CPU (central processing unit) (205) is configured to read the analog values of the sensors via analog front end conditioning electronics (204) and to transform them into predetermined values having medical interpretation relevance. Alternatively the MSD comprises resistive based sensor (209), activated by pulling out a conducting rubber plug (210), and measured by a conventional IO controller (not illustrated). The embodiment based on resistive sensor actuators may be slightly less robust, but having the advantage of being able to pose sensors on both sides of the MSD in a denser distribution (i.e. comparing to that of the inductive/capacitance sensors embodiments) without requiring the designer to deal with inductive or capacitive interference problems.

The connection to the DRI may be established using either a quick, splash and dust proof connector, for both power supply and data communication (206) or through RF NFC means and protocol, through a tuned antenna coil (207). Any power adjustments and regulation needed by the MSD electronics may be handled by e.g. a DC-DC module (208).

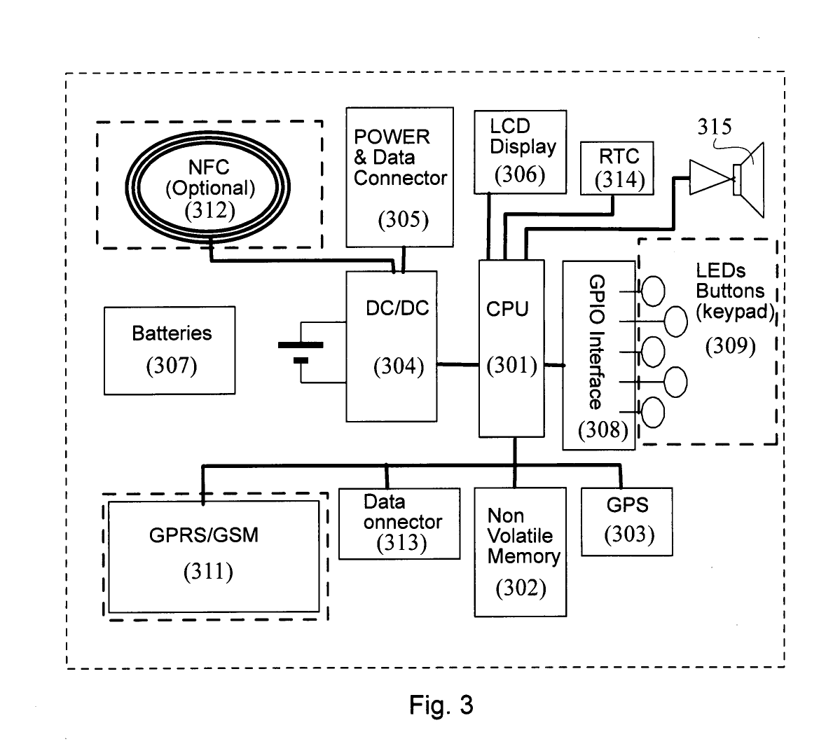

FIG. 3—depicts the Electronic Block diagram of a DRI according to various embodiments of the present invention. All the components presented in the drawing are available from various vendors. The unit consists of CPU (301) e.g. MIPS32, or an ARM9 MCU (Micro Controller Unit) able to serve a common embedded operating system e.g. Atmel AT91SAM9261 that will manage the operation of all DRI units. All data (medical status MSD records, GPS data, date and time, IDs, etc.) will be stored on non volatile Flash memory (302) e.g AT25FXXX SPI DataFlash by Atmel. User interface include LCD (any LCD type) display (306) and keypad buttons and led indicators (309) will be managed by the CPU through dedicated GPIO interface (308). An optionally provided speaker (315) will serve for transmitting audio instruction to the user. The DRI will be powered by battery (e.g. rechargeable batteries) (307). Power regulation and DC bus distribution will be handled by the DC-DC module (304). The connection to the MSD will be established using a quick and safe connector for both power supply and data communication (305) or through an optional RF NFC protocol and circuit using a tuned antenna coil (312). Preferably the unit further includes a GPS module (303) for example the NavSync's™ CW20 or the GPSM001 module from RF Solutions™, for retrieving AT time and location information and for calibrating an RTC unit (314) e.g. DS1305 by Maxim Semiconductor™ backed by a battery or by a supercapacitor. In an alternative preferred embodiment the communication with a remote server is through a GSM/GPRS cellular modem module (311) for example a GPRS modem available from Wavecom™ or Siemens™ or Telit™. In another alternative preferred embodiment, said TM is an integral part of the DRI for example a combined GPS-GPRS module e.g. GM862-GPS made by Telit™ or other GPS-cellular modem commercial module. In various preferred embodiments the DRI may be able to connect via Data Connector (313) to a third party equipment through a cable adaptor e.g. PC USB port, WLAN, and wireless modem.

A capacitive sensor according to the invention is based on the capacitance equation, according which the capacitance C is in direct ratio to the area A of the capacitor plates and in inverse ratio to their separation ‘d’. The capacitance is further affected by the relative dielectric constant ∈ᵣ of the medium separating between the plates:

$$C = \frac{{ɛ}_{0} \cdot {ɛ}_{r} \cdot A}{d}$$

Based on the above equation, the sensor traces which are printed on top of the PCB form a capacitor with a metal plate presented above (and alternatively the metal plate may serve only in changing the dielectric nature between a plurality of capacitor plates printed on the PCB). By changing the metal plate's position above the sensor, e.g. by peeling it off thereby exposing a colored spot disclosed under the metal plate for serving as said visual “reading”, the total capacitance changes as well, thus providing a twofold visual and electronically traceable change.

This change can be easily measured using any conventional capacitance measuring method (e.g. charge time measurement, charge voltage divider, frequency change by forming oscillator, or any other acceptable capacity change tracing method known in the art).

Charge Time Measurement technique for obtaining change in total capacitance:

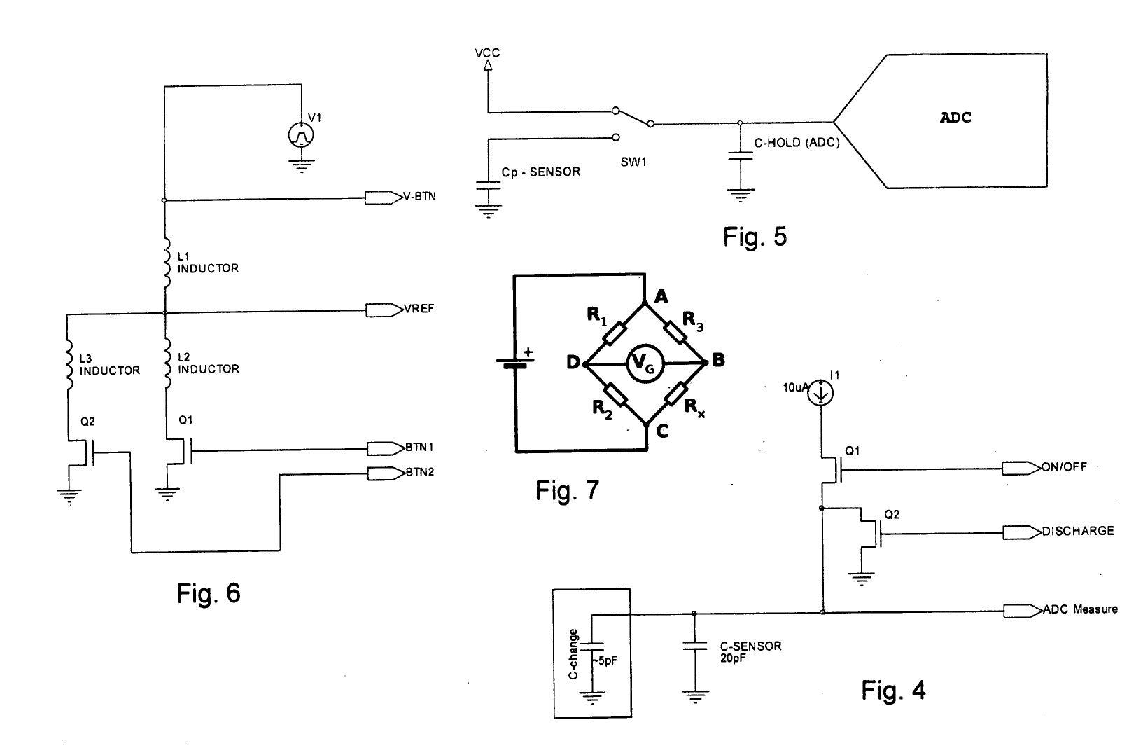

FIG. 4 exemplifies a circuit which may be constructed based on capacitor instantaneous current: I=C*dV/dt. By designing the current I to be substantially constant over time integration of both sides of said equation yields I*t=C*V. A change in the capacitance C can thus be traced by measuring the voltage V after supplying a constant current for a predetermined time ‘t’.

The circuit illustrated by FIG. 4 consists of a constant current source (e.g. 10 uA) which is controlled by and ON/OFF switch (represented here by a MOSFET transistor switch) and a DISCHARGE switch to make sure that the whole circuit is discharged before making measurements.

When no pressure is applied to the sensor, it will have a capacitance estimated in the range of 20-40 pF. When pressure is applied to distort the metal foil (and surely when the metal foil is completely peeled off from the area above the traced capacitor) a change of around 5-10 pF can be introduced to the total capacitance of the sensor. Such a change in total capacitance will cause a noticeable change in the measured voltage.

FIG. 5 illustrates a circuit making use of Charge Voltage Divider—Technique for obtaining change in total capacitance. The circuit requires no external components except a simple ADC.

By charging internal C-HOLD capacitor (of the type present in every SAR ADC in order to hold sampled signal voltage level during conversion) to VCC we set it to a known voltage level. Then, by switching SW1 the sensor capacitor Cp becomes connected in parallel with C-HOLD, thus forcing C-HOLD to loose charge for Cp. Since the voltage across Cp is proportional to its capacitance, a change in Cp is detectable by comparing its measured voltage to previous measurements taken before the change.

An inductive sensor is based on variations in total inductance of a coil due to change in proximity to metal object (similar to that of capacitive sensor presented above).

Variations of capacitance in such sensors can be measured using electronic circuit as described in FIG. 6.

A square wave generator source (V1) supplies frequency to L1 constant inductor and to L2-L3 sensors connected to the common ground through switches (Q1-Q2). When either sensor is grounded a voltage divider is formed between the constant inductor and the sensor.

By measuring RMS DC voltage it is possible to determine changes in sensor inductor values.

In an alternative embodiment, most suitable for a hermetically sealed MSD, the colored display will be generated by a chemical reaction triggered by combining two chemicals contained separately (e.g. by breaking microcapsules at least one of which containing a liquid media, and mixing their content, or e.g. with a substrate content impregnated in the button encasing material, said mixing generating a color change of said resulting mixture). The benefit of the microcapsules is that they keep the reactants away from each other until the color change is desired.

There is a great variety of color generating reactions known in the art, and maintaining the reactants in microcapsules to be smashed at the desired time is alsa known, e.g. as disclosed by US20090323066.

Electrically traceable change may be achieved in the embodiment based on chemical color change, for example by mixing a salt or metal particles into one or both color changing chemicals. Once said salt or metal particles are freed and come in contact with the substrate in the button region the metal salt or particles change the conductivity or other electrical property of said button region which is electrically measurable e.g. by measuring the change of resistance using conventional Wheatstone bridge circuit as exemplified by FIG. 7. Accordingly, simultaneous visual and electronically traceable lasting change can manually be activated in the embodiments of the invention based on chemical color change, independently of the availability of electrical power source. In an alternative embodiment, a photoelectric element (e.g. photoresistor, photodiode, phototransistor or any other device which changes its electric properties when exposed to light) is located under each of at least part of a plurality of the actuators, and a substantial change in its conductivity is detectable once the respective actuator is removed from above e.g. by measuring the change of resistance using conventional Wheatstone bridge circuit exemplified by FIG. 7 and useful for tracing changes in resistance of a photoelectric/photoresistive element as well. Of course in case photoelectric elements are used as sensors, the MSD material should be formed opaque to ambient light, and so have to be the plug to be pulled out of the device, or a foil to be peeled off a surface of the device or the coating to be scratch off, all said actuators will be opaque to ambient light including to NIR (near infrared radiation).

1. Device to be attached to a wounded person for recording and reporting medical and logistic information relating to the wounded person in mass casualty event, the device is battery free and comprises (i) a solid state memory; (ii) a plurality of actuators in a predetermined correlation with a graphical presentation presented on external surface of the device such that a status of each actuator represents a medical or logistic information relating to the wounded person; (iii) a conversion adapter configured to translate a status of the actuators into respective digital codes or to transfer the status of the actuators to external real time translator for returning the status in the form of respective digital codes wherein said codes represent medical or logistic information relating to the wounded person and are respectively loaded into the solid state memory by the conversion adapter or by the external translator; and (iv) an interface configured to communicate with a mobile electrically powered communication apparatus for extracting electrical energy required for tracing the status of the actuators, for translating it the into the digital codes and for loading them into the memory; said codes are thereby readable by the mobile communication apparatus directly from the memory for being communicated to a remote server, wherein the status of the actuators respective of the codes and in correlation with the graphical presentation presented on external surface of the device allows for immediate interpretation of the status by a human reading of the device irrespective whether the device is energized.

2. Mobile electrically powered communication apparatus configured to communicate with the device of claim 1 for temporarily energizing it and for exchanging data with the memory of the device.

3. Medical recording displaying and reporting system for displaying and electronically storing a status of a plurality of wounded persons in a mass casualty event, the system comprises a remote server capable of receiving data from the memory of each of a plurality of the device according to claim 1 through at least one mobile communication apparatus according to claim 2.

4. The device according to claim 1, wherein the actuators respond to manual actuation by electrically measurable change in resistance, conductivity, inductance or capacity or a combination thereof of respective sensors embedded in a body of the device.

5. The device according to claim 4, wherein the actuator comprise chemical reactants configured for changing the resistance of a respective sensor simultaneously with making a change in color visible in a predetermined location within the graphical presentation presented on external surface of the device, said change in color is indicative of a change in the status of the actuator.

6. The device according to claim 1, wherein the actuators comprise metal foil attached on an external surface of the device, each having a predetermined shape configured to be peeled off the surface for making electrically measurable change associated with medical or logistic information relating to the wounded person and for simultaneously making on a surface of the device a visible or tactile human interpretable change indicative of a change in the status of the actuator.

7. The device according to claim 1, wherein the actuators comprise scratch off coating in predetermined shapes and locations within the graphical presentation presented on external surface of the device, the scratch off coating has predetermined conductivity or comprises reactants in microcapsules adapted for causing electrically measurable change in resistance, conductivity, inductance or capacity or a combination of thereof of respective sensors embedded in a body of the device, once scratched off the surface of the device, and simultaneously making on the surface a visible or tactile human interpretable change indicative of a change in the status of the actuator.

8. The device according to claim 1, wherein the actuators comprise a conducting rubber plug to be pulled out of the device.

9. The device according to claim 1, wherein the actuators comprise a plug to be pulled out of the device, a foil to be peeled off a surface of the device or a scratch off coating to be scratched, said actuators are opaque to ambient light, wherein a photoelectric element is located under each of at least part of a plurality of the actuators, and a substantial change in its conductivity is detectable once the respective actuator is removed from above.

10. The device according to claim 1 wherein the actuators are disposed on the graphical presentation in repeating order, each order is associated to a same predetermined list of typical medical interventions which may be taken during a treatment of a wounded person, thereby allowing the evolving status of a wounded person over successive intervention to be visible and appreciable directly from the device without requiring a reading apparatus.

11. The device according to claim 1, further configured for mutual plugging with a communication booster module comprising an RF transceiver and battery and associated communication control circuitry, said module is thereby may optionally be connected to the MSD memory data output on site, in case an increasing communication range with the DRI is required.

12. Method for complying with medical triage, logistics and casualties transportation decision demands associated with the managing of medical support of wounded persons in mass casualty event, the method comprises equipping medical staff with (i) a plurality of medical status devices (MSDs) configured to allow for manual recordation of respectively correlated human readable and electronically traceable changes relating to medical interventions performed on or to medical measurements taken from the body of a wounded person, for accomplishing the recording of the casualties' medical status; and with (ii) at least one reading and writing device DRI configured to read information recorded on the medical status devices and to return information to enrich the information recorded on the MSD, wherein said medical status devices are attached each to a wounded person in the MCE, thereby allowing a medic treating the wounded person to record interventions and medical measurements taken to browse through the information recorded on the MSD either electronically by the DRI or visually directly from the physical status of actuation sites located on surface of the MSD.

13. The method of claim 12 wherein the DRI further comprises communication means or is capable of exchanging information with a mobile communicator in communication with remote server, thereby allowing to report the information recorded in MSDs to a remote server, thus improving the ability to comply with logistic demands associated with the managing of medical support and transportation of wounded persons in the mass casualty event.
